(19) United States
(12) Patent Application Publication
(10) Pub. No.: US 2025/0289541 A1
(43) Pub. Date: Sep. 18, 2025

(54) SUBMARINE HEATING SYSTEM, DEVICE FOR COUPLING A HEATING SOURCE TO A SUBMARINE VEHICLE AND METHOD FOR SUBMARINE HEATING

(71) Applicants: PETRÓLEO BRASILEIRO S.A. - PETROBRAS, Rio de Janeiro (BR); INSTITUTO DE ESTUDOS DO MAR ALMIRANTE PAULO MOREIRA - IEAPM, Arraial do Cabo (BR)

(72) Inventors: DANIEL ADOLPHO DA SILVA JUNIOR, Rio de Janeiro (BR); LUCIANA VICENTE RESENDE DE MESSANO, Arraial do Cabo (BR); CARLO SPEGLICH, Rio de Janeiro (BR); LUCIANA ALTVATER, Arraial do Cabo (BR); RICARDO COUTINHO, Arraial do Cabo (BR)

(73) Assignees: PETRÓLEO BRASILEIRO S.A. - PETROBRAS, Rio de Janeiro (BR); INSTITUTO DE ESTUDOS DO MAR ALMIRANTE PAULO MOREIRA - IEAPM, Arraial do Cabo (BR)

(21) Appl. No.: 19/080,250

(22) Filed: Mar. 14, 2025

(30) Foreign Application Priority Data
Mar. 14, 2024 (BR) 1020240050029

(51) Int. Cl. B63B 59/08; A61L 2/04; A61L 2/24; B08B 7/00; B08B 13/00; B63G 8/00

(52) U.S. Cl. CPC B63B 59/08; A61L 2/04; A61L 2/24; B08B 7/0071; B08B 13/00; B63G 8/001; A61L 2202/14; A61L 2202/16; B63G 2008/005

(57) ABSTRACT

The present application relates to a submarine heating system, a device for coupling a heating source to a submarine vehicle, and a method for submarine heating. In particular, the submarine heating system involves the use of a blowtorch, through a flame ignition system and a flame time and temperature control system.

### CROSS-REFERENCE TO RELATED APPLICATION

This application claims priority to Brazilian Application No. BR 1020240050029, filed on Mar. 14, 2024, the disclosure of which is herein incorporated by reference in the entirety.

### FIELD

The present application falls within the technical field of systems and methods for conserving and maintaining submarine vessels. More specifically, the present application is related to submarine heating systems and methods thereof.

### BACKGROUND

The monitoring of activities carried out in offshore environments by environmental agencies has been gaining greater notoriety in recent times, thus increasing the rigor with regard to liability for damage caused to the environment.

In particular, the conservation and maintenance of offshore structures, such as drilling or production vessels, is an activity of great relevance for environmental preservation.

For example, there is growing pressure from environmental agencies to implement control actions against bio-invasions that are located on the vessel hulls, such as the exogenous species sun coral (Tubastraea coccinea).

In this way, environmental agencies understand that the vessel hulls can act as vectors for spreading bio-invasion due to the movement of these vessels through different basins. Furthermore, the surfaces of the vessel hulls have a good capacity for the bio-invasion growth, such as sun coral, given the long time they remain stationary while drilling, storing or unloading, for example.

The need to clean a vessel's hull generates large expenditures, mobilizing other vessels, human resources and equipment.

Due to the lack of technology that allows the removal (or neutralization) of colonies in an offshore environment, the current solutions involve moving vessels to sheltered waters, where divers manually remove colony by colony from the hull.

The manual process is outdated, using hammers and metal tips, which also renders the activity dangerous.

Additionally, shallow diving activity is a work activity that presents risks. In this way, the shallow diving activity combined with the handling of blunt, cutting, and piercing tools increases the level of danger in the process of cleaning vessel cases.

Furthermore, the need for transit to sheltered waters is due to the fact that it is not possible for divers to carry out such an operation in an offshore environment.

Furthermore, the economic impact of this process stands out. The daily rates of vessels such as offshore drilling rigs make them one of the most expensive assets in the oil and gas industry. For example, the accumulated cost of moving probes to meet cleaning requirements is approximately $300 million per year.

Scientifically, it has been proven that heat has the ability to neutralize sun coral colonies.

### STATE OF THE ART

The State of the Art discloses solutions that help in the control and extermination of bio invasions, such as sun coral, as can be seen in the document of the Working Group concerning Sun Coral, Final Report (Available on the internet at https://repositorio.mcti.gov.br/handle/mctic/4696, file consulted on Nov. 1, 2024) of the Ministry of Science, Technology, Innovations and Communications (MCTIC), such as manual removal, hydrojetting, submarine techniques with surface treatment with heat and ultrasound. The analysis of this document reveals very inefficient techniques in relation to combating bio-invasions, with no specific examples being reported for combating sun coral, but rather for other biofoulings. The use of steam and mechanical removal is mentioned, but there are risks of dispersing the sun coral and increasing bio-invasions. The use of a diver with a blowtorch is also discussed, but there are risks for the diver and also limitations on autonomy regarding diving.

Furthermore, document CN103785923A discloses a local submarine welding robot via the dry method using a remotely operated vehicle (e.g., ROV). The submarine welding robot comprises a mechanical welding arm installed on the ROV. The front of the welding mechanical arm is provided with a dry method spot arc welding gun and a weld tracker. The welding robot is especially suitable for use in hard-to-reach places where divers could not access. However, this technology has not been used to combat sun coral or other fouling. Furthermore, the size of the equipment and its complexity make it very difficult to apply in combating biofouling.

Document WO2009135267A relates to an apparatus for treating a marine growth on a surface, comprising a confinement arrangement which is arranged to confine a volume of heated fluid adjacent a portion of the surface to be treated, such as a ship hull. Fluid is introduced into the volume via an entry port through a wall of the confinement arrangement. To facilitate even heating throughout the volume, the entry port is arranged so that the heated fluid is directed in a dispersed f The confinement arrangement and complexity of this solution makes it difficult to widely apply it to remove sun coral and other biofoulings. In this way, it is not reported what biofouling is or what to do with the fluid used in removal, which disposal can aggravate the problem of biofouling.

Document FR2700240B1 discloses a device for localized application of hot steam to a surface on the seabed, comprising a pressurized steam source that feeds steam through distribution pipes to the area of the seabed that requires heating. The steam is then concentrated and held in the required area by a nozzle, which can be connected to a heating chamber. The use of steam may worsen the invasion by spreading the sun coral or other biofoulings.

Furthermore, document BR 102020026998-4 reveals a robotic platform containing a flat area robot and a niche area robot, which sends the effluent from removal, capture and crushing to a modular effluent treatment system. The operations center enables the operation of all integrated systems and is housed in a support vessel, which also has an automated robot launch and recovery system, and the robotic platform control contains a software architecture capable of allowing the operator to visualize, plan and record missions. The solution reported in this reference is a very complex and laborious system to operate. Furthermore, crushing the bio-invasion organisms can worsen the problem by spreading the organisms that were not killed in this operation.

The documents cited herein do not disclose a solution to the problem of biofouling as in the present application, especially for the problem of combating sun coral. The present application herein is simple and efficient solution for the extermination of sun coral and can be applied to other biofoulings. It is worth noting that the solutions found in the state of the art can even worsen bio-invasions, since mechanical removals or the use of steam can promote the spread of sun coral or other invasive organisms, worsening the problem. Furthermore, the present application allows removal, without the use of a diver and with a heat source, in complex locations, such as the removal of biofouling from propellers and propeller supports (thruster) not reported in the state of the art.

### SUMMARY

The present application, according to a preferred embodiment thereof, defines a submarine heating system comprising: at least one blowtorch, at least one support for spark flame ignition, at least one ignition device, at least one fastening tool, at least one control system, at least one source of O₂, at least one LPG source, at least one LPG regulator and at least one O₂ regulator. The submarine heating system is fixed to the side of a submarine vehicle by means of at least one fastening tool.

The at least one blowtorch is used with the suppression of at least one cutting O₂ source.

The at least one spark flame ignition support is controlled by radio frequency.

Furthermore, the submarine heating system additionally comprises at least one O₂ hose and at least one LPG hose such that, there is at least one source of O₂ which includes at least one cylinder of O₂; and there is at least one LPG source, which includes at least one LPG cylinder.

The at least one ignition device comprises at least one ignition coil.

The at least one fastening tool is fixed to at least one base of the submarine vehicle by means of at least one fastening means.

The submarine heating system, characterized in that the submarine vehicle can be a miniature remotely operated vehicle (e.g., miniROV). The miniROV, when compared to the ROV, is a smaller equipment and that is much simpler to use, and fits in a backpack, for example. The ROV is a much larger equipment designed for operations that require a support boat. Furthermore, the size of the ROV does not allow movements in complex and difficult to access locations, such as, for example, the removal of biofouling on the propellers and propeller supports, called thruster.

The submarine heating system further comprises at least one human-machine interface connected to at least one controller, which is connected to at least one control system. The at least one controller comprises at least one programmable logic controller. The human-machine interface presents flame temperature and time information.

There is a connection between at least one human-machine interface with at least one controller; and there is a connection between at least one controller to at least one control system which may be a wired or wireless connection.

Furthermore, the submarine heating system further comprises at least one solenoid valve. The at least one solenoid valve is derived via at least one human-machine interface, which sends a signal to at least one controller, which is connected to at least one control system, which activates and controls via at least one ignition device, which drives at least one support for spark flame ignition. The at least one control system controls the flame temperature and time.

Complementarily, the submarine heating system additionally comprises a chamber and a plurality of protection rods arranged at one end of at least one fastening tool.

Furthermore, the present invention, according to another preferred embodiment thereof, defines a device for coupling a heating source to a submarine vehicle comprising at least one support for spark flame ignition, at least one ignition device, and at least one fastening tool.

Furthermore, the heating source comprises at least one blowtorch. The heating source is attached to at least one fastening tool, and the at least one fastening tool is attached to a submarine vehicle.

Additionally, the device for coupling a heating source to a submarine vehicle further comprises at least one camera and a plurality of protective rods arranged at one end of at least one fastening tool. The at least one support for spark flame ignition is controlled by radio frequency. The at least one ignition device comprises at least one ignition coil. The submarine vehicle may be a miniROV.

Additionally, according to a preferred embodiment, the present application defines a method for submarine heating using a submarine heating system, comprising the steps of: driving the submarine heating system by driving at least one solenoid valve via at least one human-machine interface, inserting a flame time interval in at least one human-machine interface such that, the at least one human-machine interface sends a signal to at least one controller, which is connected to at least one control system, which drives at least one ignition coil, which drives at least one spark flame ignition support, and controlling the temperature and flame time, through the control system.

### BRIEF DESCRIPTION OF THE DRAWINGS

The present invention will be described below with reference to its typical embodiments and with reference to the attached drawings, although not limiting, in which:

FIG. 1 shows an illustrative diagram of a submarine heating system.

FIGS. 2A-2B illustrate an attachment schematic of a submarine heating system to a submarine vehicle.

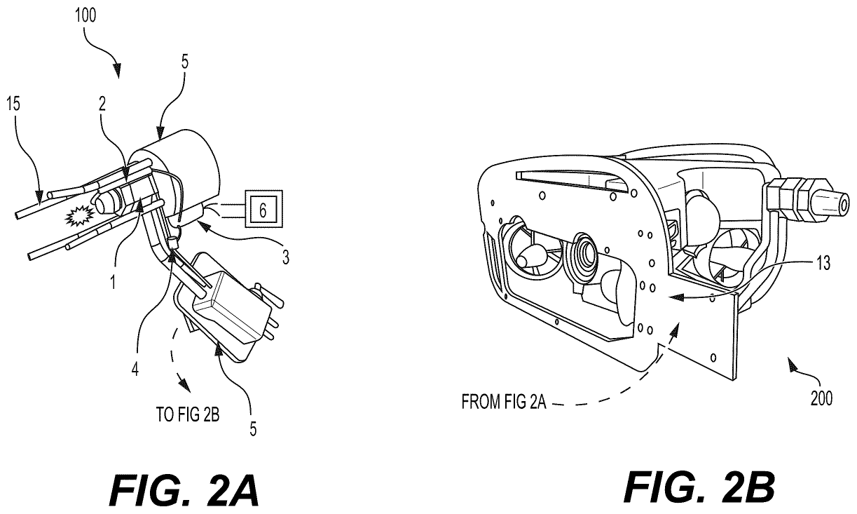

FIG. 3 shows a submarine heating system fixed to a submarine vehicle.

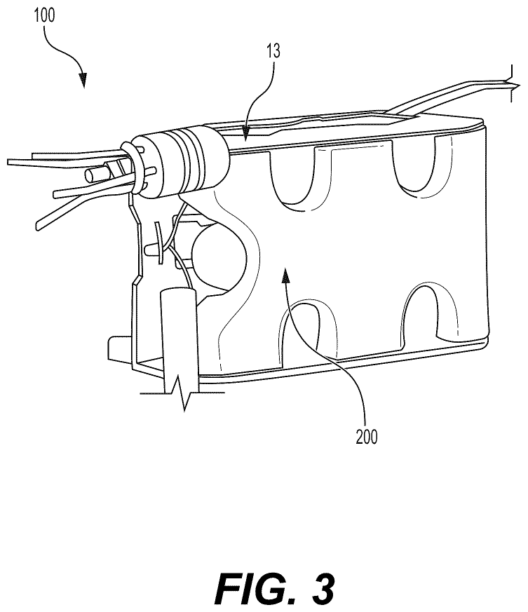

FIG. 4 represents a submarine heating system and a control system.

FIG. 5 illustrates a device for coupling a heating source to a submarine vehicle.

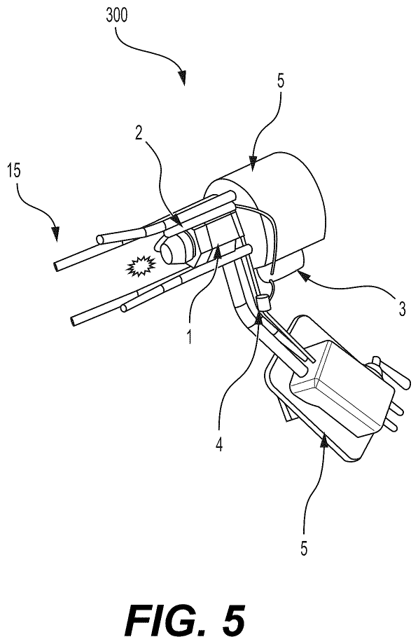

### DETAILED DESCRIPTION

Specific embodiments of the present disclosure are described below. In an effort to provide a concise description of these embodiments, all features of an actual implementation may not be described in the specification. It should be appreciated that in the development of any actual implementation, as in any engineering or design project, numerous implementation-specific decisions must be made to achieve the specific objectives of the developers, such as compliance with system-related and business constraints, which may vary from one implementation to another. In addition, it should be appreciated that such a development effort may be complex and time-consuming, but would nevertheless be a routine design and manufacturing undertaking for those of ordinary skill having the benefit of this disclosure.

Accordingly, certain details may be omitted from the description that follows with the understanding that the technician skilled on the subject has prior knowledge to fill these gaps. For example, the specific types of used devices or underlying physical principles may be omitted without impairing the description of the present invention.

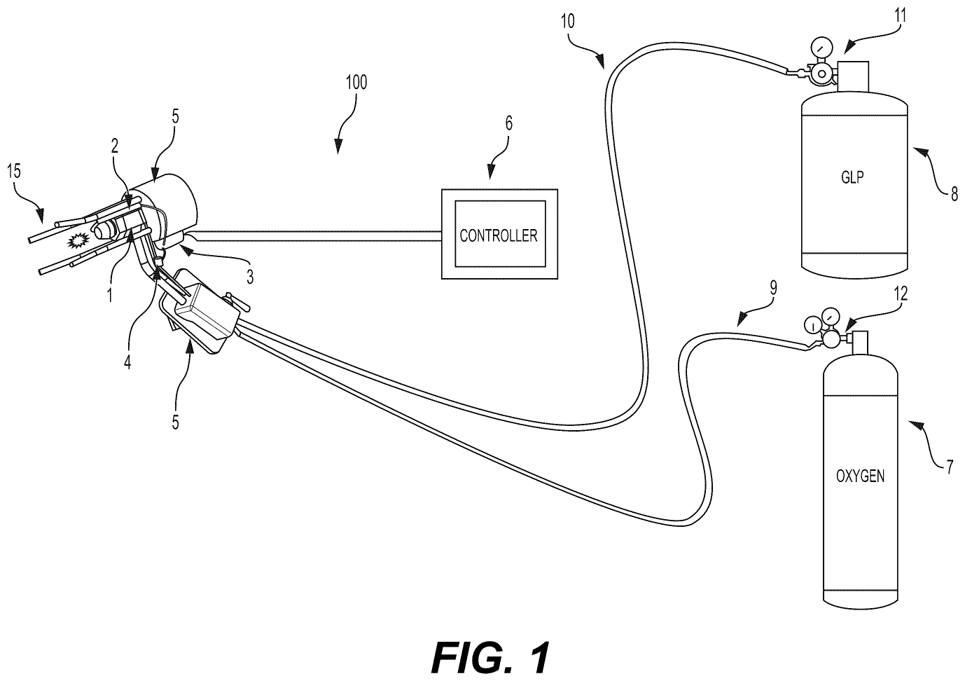

As illustrated in FIG. 1, which presents a schematic of the submarine heating system 100, according to a preferred embodiment of the present invention, the submarine heating system 100 comprises: at least one blowtorch 1, at least one spark flame ignition support 2, at least one ignition device 3, comprising at least one ignition coil 3, at least one metal clamp 4, at least one fastening tool 5, at least one control system 6, at least one O₂ source 7, at least one LPG source 8, at least one O₂ hose 9, at least one LPG source 10, at least one LPG regulator 11, at least one O₂ regulator 12, or a combination thereof.

In some embodiments, there is at least one O₂ source 7 which includes at least one O₂ cylinder 7, and there is at least one LPG source 8 that includes at least one LPG cylinder 8.

In particular, the submarine heating system 100 is attached to the side of a submarine vehicle 200, by means of at least one attachment tool 5. In some embodiments, the submarine vehicle may be a miniROV 200.

In some embodiments, there is at least one fastening tool 5 fixed, by at least one fastening means, on at least one base 13 of the submarine vehicle 200, which can be a base 13 of polymeric material arranged on the submarine vehicle 200. A fastening means comprises at least one screw or at least one clamp or at least one fastening means which may be welded or any other compatible type of fastening.

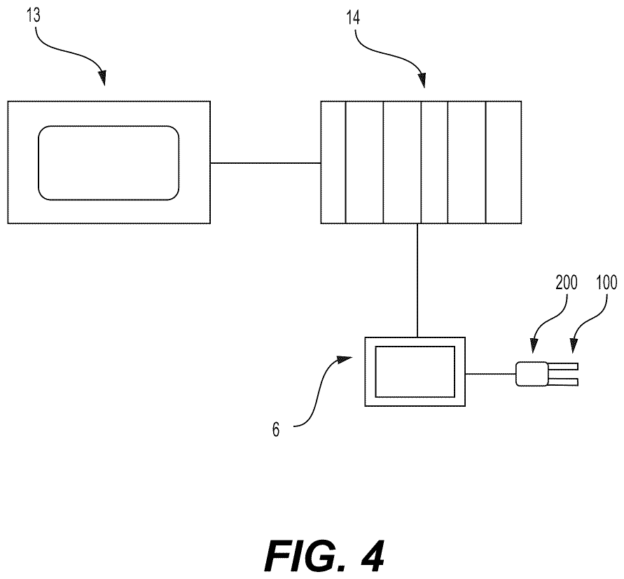

As seen in FIG. 4, the submarine heating system 100 further comprises at least one human-machine interface 13, connected to at least one controller 14, which is connected to at least one control system 6. The at least one controller 14 comprises at least one programmable logic controller (PLC).

In particular, the connection between at least one human-machine interface 13 with at least one controller 14; and the connection between at least one controller 14 to at least one control system 6 may be a cable or wireless connection.

According to a particular embodiment, a software control or a set of instructions is applied to a programmable logic controller 14, providing temperature measurement at the surface or as close as possible to the sun coral, in a closed loop system, attesting to the annihilation of the sun coral. The software is able to combine the flame time and the measured temperature into an image, which proves the annihilation of the sun coral.

Furthermore, according to another preferred embodiment of the present invention, the submarine heating system 100 additionally comprises at least one solenoid valve.

In this sense, according to another preferred embodiment of the present invention, the flame in the blowtorch 1 is generated by driving at least one solenoid valve, based on the indication of the flame time in the at least one human-machine interface 13, which sends a signal to at least one controller 14, which is connected to the at least one control system 6, which activates and controls at least one ignition coil 3, which drives at least one support for spark flame ignition 2.

At least one control system 6 controls the flame temperature and time.

Information such as flame temperature and time, and a data logging function (data logger) can be accessed by the user in at least one human-machine interface 13.

In particular, at least one spark flame ignition support 2 is radio frequency controlled.

Additionally, the submarine heating system 100 may include a camera. The camera sends images to the HMI display.

Furthermore, the submarine heating system 100 further comprises a plurality of protective rods 15, wherein the protective rods 15 serve to prevent collision of the heating source or blowtorch with a subsea structure. The plurality of protective rods 15 are arranged at one end of the at least one clamping tool 5.

According to the submarine heating system 100 of the present invention, another factor taken into consideration is the use of a flame ignition system, although with a principle similar to the spark stove, it required the use of a more powerful coil and a control system 6, which can be connected via a wireless network or using an electric cable to the submarine heating system 100 of the present invention.

In the submarine heating system 100 of the present invention, at least one blowtorch 1 is used with the suppression of the cutting O₂ source 7, that is, at least one blowtorch 1 is used without activating the cutting oxygen jet valve. Thus, it is possible to heat the surface of a vessel's hull, such as a drilling rig.

One of the challenges of using blowtorches on a submarine vehicle, such as a miniROV, for submarine heating is the operational difficulty impacted by the geometry of the part and buoyancy; these factors make maneuverability very difficult.

Additionally, the present invention defines, according to another embodiment thereof, a device for coupling a heating source 1 to a submarine vehicle 300, wherein the heating source comprises: at least one blowtorch 1, at least one spark flame ignition support 2, at least one ignition device 3 comprising at least one ignition coil 3, at least one metal clamp 4 and at least one fastening tool 5. The heating source 1 is fixed to the at least one clamping tool 5. The at least one fastening tool 5 is attached to a submarine vehicle 200. The at least one spark flame ignition support 2 is controlled by radio frequency. The submarine vehicle can be a miniROV 200.

Additionally, the device for coupling a heating source to a submarine vehicle 300 may include a camera.

The device for coupling a heating source to a submarine vehicle 300 further comprises a plurality of protective rods 15, such that the protective rods 15 serve to prevent collision of the heating source or blowtorch with a submarine structure. The plurality of protective rods 15 are arranged at one end of the at least one clamping tool 5.

Furthermore, the present invention also defines a method for submarine heating using a submarine heating system 100 as described above, comprising the steps of: driving the submarine heating system 100 by driving at least one solenoid valve via at least one human-machine interface 13, inserting a flame time interval in at least one human-machine 13 interface 13 such that, the at least one human-machine interface 13 sends a signal to at least one controller 14, which is connected to at least one control system 6, which drives at least one ignition coil 3, which drives at least one spark flame ignition support 2, and controlling the temperature and flame time, through the control system 6.

Tests have shown that a colony of sun coral measuring, for example, 10 cm³ of volume was obliterated in 3 seconds. In other words, there was total extermination of the sun coral. It is worth noting that there was no spreading of the sun coral, as can occur with mechanical removal techniques or the use of steam, which would propagate bio invasion. Furthermore, the invention is simple and efficient when compared with the state of the art. The application depth of the invention is at least 50 m, dispensing the use of divers or complex apparatus such as those disclosed in the state of the art.

Those skilled in the art will value the knowledge presented herein and will be able to reproduce the invention in the presented embodiments and in other variants, covered in the scope of the appended claims.

1. A submarine heating system, the system comprising:
at least one blowtorch;
at least one spark flame ignition support;
at least one ignition device;
at least one fastening tool;
at least one control system;
at least one O₂ source;
at least one liquefied petroleum gas (LPG) source;
at least one LPG regulator;
at least one O₂ regulator; and
wherein the submarine heating system is fixed to a side of a submarine vehicle using the at least one fastening tool.

2. The system of claim 1, wherein the at least one blowtorch is used with suppression of at least one cutting O₂ source.

3. The system of claim 1, wherein the at least one spark flame ignition support is controlled by radio frequency.

4. The system of claim 1, further comprising:
at least one O₂ hose and at least one LPG hose;
wherein there is at least one O₂ source including at least one O₂ cylinder; and
wherein there is at least one LPG source including at least one LPG cylinder.

5. The system of claim 1, wherein the at least one ignition device comprises at least one ignition coil.

6. The system of claim 1, wherein the at least one fastening tool is fixed to at least one base of the submarine vehicle, through at least one fastening means.

7. The system of claim 1, wherein the submarine vehicle is a miniROV.

8. The system of claim 1, further comprising:
at least one human-machine interface connected to at least one controller;
wherein the at least one controller is connected to the at least one control system;
wherein at least one controller comprises at least one programmable logic controller (PLC); and
wherein the human-machine interface presents flame temperature and time information.

9. The system of claim 8, wherein the connection between the at least one human-machine interface with the at least one controller and the connection between the at least one controller to the at least one control system is a cable or wireless connection.

10. The system of claim 8, further comprising:
at least one solenoid valve;
wherein the at least one solenoid valve is derived via the at least one human-machine interface, which sends a signal to at least one controller, which is connected to at least one control system, which activates and controls the at least one ignition device, which drives the at least one spark flame ignition support; and
wherein the at least one control system controls the flame temperature and time.

11. The system of claim 1, further comprising a camera.

12. The system of claim 1, further comprising:
a plurality of protective rods arranged at one end with the at least one fastening tool.

13. A device coupling a heating source to a submarine vehicle, the device comprising:
at least one spark flame ignition support;
at least one ignition device; and
at least one fastening tool.

14. The device of claim 13, wherein the heating source comprises at least one blowtorch, wherein the heating source is fixed to at least one fastening tool, and wherein at least one fastening tool is attached to a submarine vehicle.

15. The device of claim 13, further comprising at least one camera.

16. The device of claim 13, wherein the at least one spark flame ignition support is controlled by radio frequency.

17. The device of claim 13, wherein the at least one ignition device comprises at least one ignition coil.

18. The device of claim 13, wherein the submarine vehicle is a miniROV.

19. The device of claim 13, further comprising:
a plurality of protective rods arranged at one end with at least one fastening tool.

20. A method for submarine heating using the submarine heating system of claim 1, the method comprising:
driving the heating submarine system by driving at least one solenoid valve via at least one human-machine interface;
inserting a flame time interval in the at least one human-machine interface, wherein at least one human-machine interface sends a signal to at least one controller, which is connected to at least one control system, which activates at least one ignition coil, which drives the at least one spark flame ignition support; and
controlling a temperature and flame time, through the at least one control system.
